(19) United States
(12) Patent Application Publication
(10) Pub. No.: US 2019/0041387 A1
(43) Pub. Date: Feb. 7, 2019

(54) DIGITAL BIOSENSOR

(71) Applicant: Spin-Darc LLC, Raleigh, NC (US)

(72) Inventor: Jeffery R.X. Auld, Raleigh, NC (US)

(21) Appl. No.: 16/050,460

(22) Filed: Jul. 31, 2018

Related U.S. Application Data
(60) Provisional application No. 62/542,098, filed on Aug. 7, 2017.

(51) Int. Cl. G01N 33/543; G01N 33/487; G01N 1/28; G01N 1/36

(52) U.S. Cl. CPC G01N 33/54373; G01N 33/48707; G01N 1/2813; G01N 1/40; B01J 2219/00605; B01J 2219/00702; G01N 33/54393; G01N 1/36

(57) ABSTRACT

The presently disclosed subject matter relates to a biodetection system centered on the development and optimization of a logistically simple assay for detecting, identifying, and/or quantifying microbial pathogens using an unmodified substrate. Specifically, the disclosed system quantitatively measures target analytes (e.g., bacteria) isolated over a digitally-encoded substrate. Isolated microbes are positioned in specific locations and geometries on the substrate data surface, resulting in a discernible interruption and/or change to data being read from the substrate. The change can be exploited to indicate positive detection and detection counts for one or more specific microbes.

### CROSS REFERENCE TO RELATED APPLICATIONS

This application claims the benefit of U.S. Provisional Patent Application No. 62/542,098 filed Aug. 7, 2017, incorporated by reference herein in its entirety.

### TECHNICAL FIELD

The presently disclosed subject matter relates generally to digital biosensors and to methods of using the disclosed biosensors to detect the presence of one or more analytes.

### BACKGROUND

Microbial infections pose serious risks to public health throughout the world. The Center for Disease Control and Prevention (CDC) estimates that each year, 48 million people in the United States get sick from foodborne illnesses caused by pathogenic bacteria, with Campylobacter and Salmonella reported as the most common species causing infection. Foodborne and waterborne infections remain a persistent threat to the health and readiness of the U.S. armed forces. Additionally, sexually transmitted infections (STIs), particularly, Chlamydia trachomatis (CT) and Neisseria gonorrhoeae (NG), have shown a marked annual increase. CT and NG are among the most common causes of STI and may have serious reproductive health consequences beyond the immediate impact of the infection itself (e.g., infertility or mother-to-child transmission). The Centers for Disease Control and Prevention (CDC) report an unprecedented rise of STIs in the U.S. Chlamydia infections lead the way in terms of sheer numbers, with more than 1.6 million new cases reported in 2016. Undiagnosed STIs account for nearly $16 billion in healthcare costs annually. In many cases, low-income and disadvantaged populations may be among those at greatest risk, while their access to laboratory diagnostics may be most limited. Unfortunately, the reports of growing disease burden come at a time when state and local STI prevention programs are experiencing budget cuts, forcing reductions in clinic hours and availability of screening for common infections. With increased prevalence of STIs and with resources for their diagnosis and treatment stretched thin, rapid, accurate, and affordable methods of POC screening for STIs are urgently needed. The majority of microbial infections are most effectively treated when diagnosed early, besides, early and accurate detection of causative pathogens may reduce the inappropriate use of antibiotics. Therefore, rapid, reliable, and inexpensive microbiology testing methods usable at the point of care (POC) or in low-resource settings have been the focus of industry innovation and government research.

Existing biosensor systems, such as molecular diagnostics, can be expensive and often require advanced technical expertise to operate safely and effectively. Further, current bio-detection technology requires extensive sample preparation and/or culturing, which are time-consuming and thus not feasible in emergency situations or in low-resource/POC environments. In addition, poor infrastructure, inconsistent supply chains, and insufficient personnel training limit the accessibility and performance of existing bio-detection methods on a large scale.

Optical digital storage methods (such as the rotating electromagnetic disks, e.g., DVDs) use electromagnetic radiation to read digital information encoded on a surface and provide a convenient way to store digital information in a portable device. Devices for reading and interpreting the information stored on rotating electromagnetic disks are very common, with many individuals having ready access to a number of different devices for reading the information. These common devices can resolve the sub-micrometer structures for storing data and can therefore also be used to detect tagged analytes of similar or larger scale.

Therefore, it would be beneficial to provide a method and system that enables rapid, easily deployable, cost-effective, and reliable detection and identification of pathogenic microorganisms by applying an optical digital storage method, such as the electromagnetic sensor disk.

### SUMMARY

In some embodiments, the presently disclosed subject matter is directed to a digital biosensor for detecting the presence or amount of one or more analytes in a sample. The digital biosensor comprises an optically read digital substrate that includes a top face with a layer comprising a data path capable of being read by an optical drive incident upon the layer, wherein the data path is encoded with a baseline data that is static, and wherein the top face is defined by an assay surface used for sample deposition. The biosensor further comprises a cover that can be overlaid on the top face of the digital substrate, wherein the cover attaches to the top face about a circumference of the digital substrate. In some embodiments, the cover further comprises a centrally-positioned aperture that aligns with a centrally-positioned aperture in the digital substrate. The digital biosensor is configured such that an amount of the one or more analytes can be positioned between the top face of the digital substrate and the cover. In other embodiments, the sample can be deposited on the inside surface of the cover, with the digital substrate placed on top of it, top face down. The presence, amount, or both of the one or more analytes can be detected by an interruption or change to the baseline data being read from the digital substrate by the optical drive.

In some embodiments, the digital substrate is a digital optical disk, such as a DVD. In some embodiments, the digital substrate is constructed from one or more hydrophobic polymers selected from polyethylene, polyvinyl chloride (PVC), polystyrene, high impact polystyrene (HIPS), polypropylene, polyester, polyacryolonitrile (PAN), ethylcellulose, cellulose acetate, methacrylate, polycarbonate, acrylic acid copolymer, acrylate, or combinations thereof.

In some embodiments, the analyte is selected from the bacteria, viruses, fungi, spores, or combinations thereof.

In some embodiments, the cover is constructed from polycarbonate.

In some embodiments, the cover is attached to the top face via an outer ring positioned about the circumference thereof.

In some embodiments, the top face of the digital substrate comprises a plurality of uniformly distributed data pits and lands. In some embodiments, the pits have a width of about 0.3 μm separated by lands with a width of about 0.7 μm.

In some embodiments, the presently disclosed subject matter is directed to a system for preparing a digital bioassay. The system comprises a base with a top face defined by a hub extending upwards therefrom and a magnet positioned adjacent to the top face, wherein the hub is configured to maintain a digital biosensor on the base by cooperating with the central apertures of a cover and the digital substrate.

In some embodiments, the magnet is selected from an electromagnet or a fixed magnet.

In some embodiments, the presently disclosed subject matter is directed to a method of detecting the presence or amount of an analyte in a liquid sample. The method comprises depositing a volume of the sample within a container and adding a volume of magnetic beads with an attached capture probe to the container, wherein the capture probe is selected to bind the target analyte. The method further comprises contacting the exterior of the container with a magnet to immobilize the analyte-bound magnetic beads in one area of the container. The method includes removing the excess liquid from the container, leaving the analyte-bound magnetic beads within the container, removing the magnet from contact with the exterior of the container, and re-suspending the analyte-bound magnetic beads. The method comprises positioning a digital substrate on a base with a top face defined by a hub extending upwards therefrom and a magnet positioned adjacent to the top face, wherein the hub is configured to maintain the digital substrate on the base by cooperating with the central apertures of a cover and the digital substrate. The method comprises depositing the re-suspended analyte-bound magnetic beads onto a top face of the digital substrate, so that the magnet of the base immobilizes the analyte-bound magnetic beads, allowing for the removal of suspension fluids from the sample. The method includes overlaying a cover on the top face of the digital substrate, wherein the cover is attached to the top face about a circumference of the digital substrate to thereby create a digital biosensor assay assembly with analyte-bound magnetic beads positioned between the top face of the digital substrate and the cover; wherein the top face of the digital substrate comprises a data path capable of being read by an optical drive incident upon the layer, wherein the data path is encoded with a baseline data that is static. The method comprises removing the digital biosensor from the system and reading the digital biosensor on an optical drive, wherein the presence or amount of the analyte is detected by an interruption or change to the baseline data being read from the digital substrate by the optical drive.

In some embodiments, the top face of the base further comprises a hub extending upwards therefrom, wherein the hub is configured to maintain the digital biosensor on the base.

In some embodiments, the magnetic beads are selected from ferromagnetic beads, paramagnetic beads, or combinations thereof.

In some embodiments, the analyte is selected from the bacteria, viruses, fungi, spores, or combinations thereof.

In some embodiments, detecting the presence or amount of an analyte in a liquid sample is performed in 1 hour or less.

### BRIEF DESCRIPTION OF THE DRAWINGS

The previous summary and the following detailed descriptions are to be read in view of the drawings, which illustrate some (but not all) embodiments of the presently disclosed subject matter. Like numbers refer to like elements throughout the specification and drawings.

FIG. 1 is a front plan view of a base comprising a biosensor in accordance with some embodiments of the presently disclosed subject matter.

FIG. 2a is a perspective view of a digital substrate in accordance with some embodiments of the presently disclosed subject matter.

FIG. 2b is a magnified representation of the top surface of the digital substrate of FIG. 2a.

FIG. 3a is a top plan view of a cover that can be used with a biosensor in accordance with some embodiments of the presently disclosed subject matter.

FIG. 3b is a top plan view of a digital substrate comprising a cover in accordance with some embodiments of the presently disclosed subject matter.

FIG. 4a is a schematic illustrating the deposit of a sample comprising an analyte into a container in accordance with some embodiments of the presently disclosed subject matter.

FIG. 4b is a schematic illustrating the addition of magnetic beads comprising a capture probe to the sample of FIG. 4a in accordance with some embodiments of the presently disclosed subject matter.

FIG. 4c is a schematic illustrating the immobilization of the analyte-bound magnetic beads of FIG. 4b in accordance with some embodiments of the presently disclosed subject matter.

FIGS. 4d and 4e are schematics illustrating depositing re-suspended analyte-bound magnetic beads onto the top surface of a digital substrate in accordance with some embodiments of the presently disclosed subject matter.

FIG. 5a is a front plan view of a digital substrate positioned on the top face of a base in accordance with some embodiments of the presently disclosed subject matter.

FIG. 5b is a front plan view of the digital substrate of FIG. 5a after re-suspended analyte-bound magnetic beads have been deposited on the top surface in accordance with some embodiments of the presently disclosed subject matter.

FIG. 5c is a front plan view of the digital substrate of FIG. 5b after fluid has been removed in accordance with some embodiments of the presently disclosed subject matter.

FIG. 5d is a representation of analyte-bound magnetic beads positioned on the top surface of the digital substrate in accordance with some embodiments of the presently disclosed subject matter.

FIG. 5e is a magnified view of the analyte-bound magnetic beads of FIG. 5d.

FIG. 5f is a front plan view of a digital substrate and cover positioned on a base in accordance with some embodiments of the presently disclosed subject matter.

FIG. 5g is a front plan view of the digital substrate and cover assembly of FIG. 5f being removed from the base in accordance with some embodiments of the presently disclosed subject matter.

FIG. 5h is a side plan view of a digital substrate comprising a cartridge in accordance with some embodiments of the presently disclosed subject matter.

FIG. 5i is a top plan view of the cartridge of FIG. 5h.

FIG. 6 is a line graph illustrating testing of the PI error of paramagnetic microbeads using 10 samples.

FIG. 7 is a line graph illustrating the log DVD read error rate versus log E. coli concentration.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments+/−20%, in some embodiments+/−10%, in some embodiments+/−5%, in some embodiments+/−1%, in some embodiments+/−0.5%, and in some embodiments+/−0.1%, from the specified amount, as such variations are appropriate in the presently disclosed subject matter.

The presently disclosed subject matter relates to a bio-detection system centred on the development and optimization of a rapid, low cost, and logistically simple assay for detecting, identifying, and/or quantifying microbes (e.g., bacterial pathogens) using an unmodified substrate. Specifically, the disclosed system isolates analytes (e.g., microbial targets) in controlled geometries over a hydrophobic digitally-encoded substrate. Isolated microbes are positioned in specific locations and geometries on the substrate data surface, resulting in a discernible interruption and/or change to data being read from the substrate. The change can be exploited to indicate positive detection and/or provide detection counts for one or more specific microbes. The disclosed microbial detection system and methods therefore enable virtually any computer with a built-in or portable optical drive to perform rapid, highly sensitive, and highly specific assays.

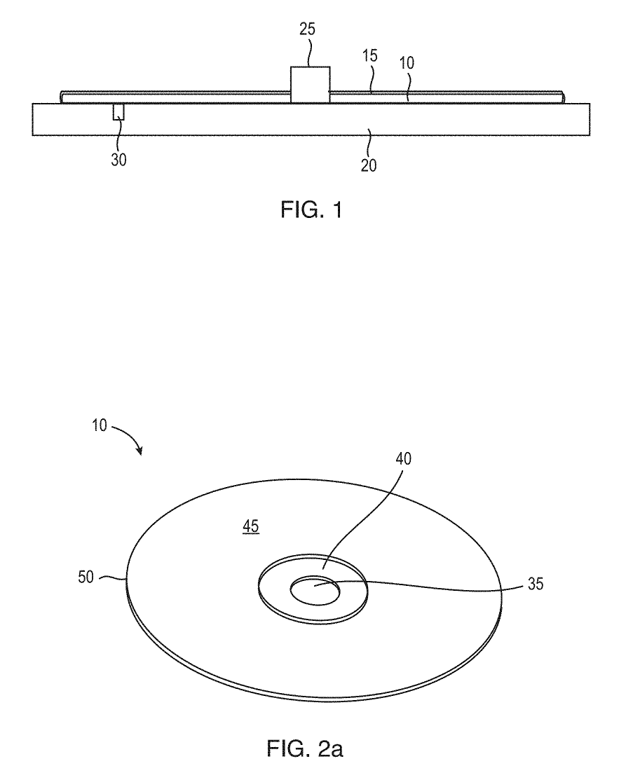

As shown in FIG. 1, the disclosed bio-detection system comprises an optically read digital substrate 10 that functions as an assay surface for sample deposition. The system includes base 20 that provides a support surface for the substrate and hub 25 that extends upward form the base and prevents the substrate from moving, such as during preparation and separation of analyte samples. Magnet 30 is positioned directly below the substrate application site. Cover 15 is positioned over substrate 10, thereby encapsulating the substrate. The substrate-cover complex as a unit can be positioned into an optical disk drive to perform the detection assay, as set forth in more detail herein below.

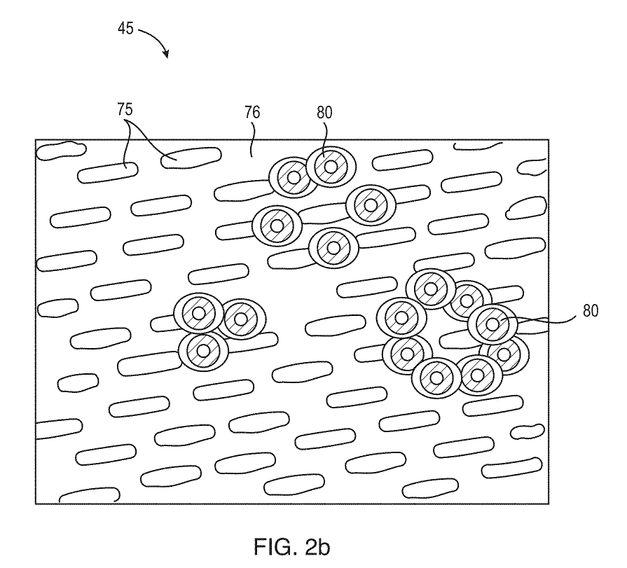

FIG. 2a illustrates one embodiment of optically read digital substrate 10 configured as an unmodified optical biosensor disk. In some embodiments, the substrate can be configured as a DVD. However, it should be appreciated that the disclosed optically read digital substrate is not limited and can include any digital storage device that can support the presence of one or more bio-entities while being processed. For example, suitable substrates can include (but are not limited to) carrier films for magnetic tapes, photoresists and electron beam resists, Blu-Ray disks, and supports used for magnetic and optical discs for video replication and data storage. In some embodiments, storage systems with high packing densities can be used, such as magneto-optical systems, phase change systems, memories based on photopolymers, and/or polymers with liquid crystalline side chains.

Substrate 10 can be constructed from one or more hydrophobic polymers used both as components and actual storage materials. The term “hydrophobic” as used herein refers to a surface or material that exhibits water-repelling properties. Any suitable hydrophobic polymer can be used, including (but not limited to) polyethylene, polyvinyl chloride (PVC), polystyrene, high impact polystyrene (HIPS), polypropylene, polyester, polyacryolonitrile (PAN), cellulose derivatives (such as ethylcellulose or cellulose acetate), methacrylate, polycarbonate, acrylic acid copolymer, acrylate, and combinations thereof. In some embodiments, the material used to construct substrate 10 can be selected to ensure that there is no interference with the signal return in an optical drive during testing. For example, material having a Refractive Index of 1.51 would be highly compatible with the polycarbonate material used in the manufacture of several specific digital optical substrates.

Substrate 10 can be configured in a number of different sizes, shapes, and configurations. In some embodiments, substrate 10 includes an aligned center aperture 35 sized and shaped to allow the substrate to fit over hub 25 of base 20, as shown in FIG. 2a. Center aperture 35 can be constructed with any desired diameter, such as about 10-20 mm (e.g., about 10, 11, 12, 13, 14, 15, 16, 17, 18, 19, or 20 mm). Moving outward from the center aperture on the surface of the substrate is transition area 40 that serves as a buffer between center aperture 35 and assay region 45. The transition area can have a diameter of about 44 mm (e.g., 35, 36, 37, 38, 39, 40, 41, 42, 43, 44, 45, 46, 47, 48, 49, 50, 51, 52, 53, 54, or 55 mm). Assay region 45 lies between transition area and outer edge 50 of the substrate. The assay region is suitable for the attachment of one or more biostructures (such as analyte-bound magnetic beads). A standard optical drive can rapidly scan assay region 45 with a visible wavelength laser to detect patterns of reflected light. By precisely immobilizing microbial targets capable of scattering laser light on the surface of assay region 45, the disclosed assay functions as a low-cost optical biosensor.

Assay region 45 of the substrate comprises continuous data spiral 55 that extends from the edge of the transition area and spirals outward toward outer edge 50. A single physical track 60 is defined as one complete turn (360 degrees) of the continuous data spiral. The track-to-track separation of a standard DVD is about 0.7 μm. However, the track-to-track separation 65 of substrate 10 is not limited and can be larger or smaller depending on the type of device used to read the substrate, the type of analyte being detected, etc.

As shown in FIG. 2b, substrate assay region 45 comprises a plurality of data pits 75 and lands 76 formed along the continuous data path using any known method. For example, in some embodiments, the pits and lands can be formed when the substrate is initially fabricated by pressing or casting. Alternatively, the pits and lands can be formed using microelectronic and microfabrication techniques, photolithography, sputtering, chemical vapor deposition, deep reactive ion etching, wet and dry etching, and the like. The term “pits” refers to depressions in the surface of the substrate surrounded by the other surface of the face of the substrate, commonly called “lands.”

Thus, the continuous data path can be formed as an array of pits and lands constructed in the substrate. In some embodiments, the array of pits and lands can be coated with a reflective surface (e.g., silver, aluminium, gold, copper) such that when the focused electromagnetic radiation contacts a particular spot, the focused radiation is substantially reflected from the reflective surface as reflected radiation. The reflective surface can be selected by one of ordinary skill in the art to achieve the necessary reflection of the focused radiation necessary to detect the change due to analyte present in structures encoded on the continuous data path.

The depth of each pit can be nominally the quarter wave distance of the focused electromagnetic radiation. The depth of the pit causes destructive interference with the reflected radiation, thereby reducing the overall intensity of the reflected radiation. The reduction in intensity causes the radiation detector to read an average decrease in radiation at the reflected spot that allows the system to differentiate between a pit and a land.

As shown in FIG. 2b, in some embodiments, the pits can be configured as substantially elliptical depressions formed in the surface of substrate 10. The major axis of each elliptical depression can be oriented along the physical track. The difference in height between the pits and lands cause the intensity of the reflected radiation to vary based on whether the focused radiation falling on a specific spot is reflected as higher or lower intensity reflected radiation. The difference in intensity of the radiation allows the disk system to determine whether the spot is falling on a pit or a land area of the biosensor disk.

In some embodiments, pits 75 can be configured with a width of about 0.3 μm (e.g., no more/less than about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1 μm) separated by lands 76 of about 0.7 μm (e.g., no more/less than about 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, or 1.5 μm). As such, the substrate provides a dense, readable submicron platform suitable for the detection of one or more microbes 80 overlaying data pits 75 and/or lands 76.

Importantly, no chemical patterning is used in preparing substrate 10 for the disclosed assay. “Chemical patterning” refers to the creation of a geometric or topological pattern of chemical entities or groups on the surface of the substrate, in order to immobilize target analytes from a deposited sample.

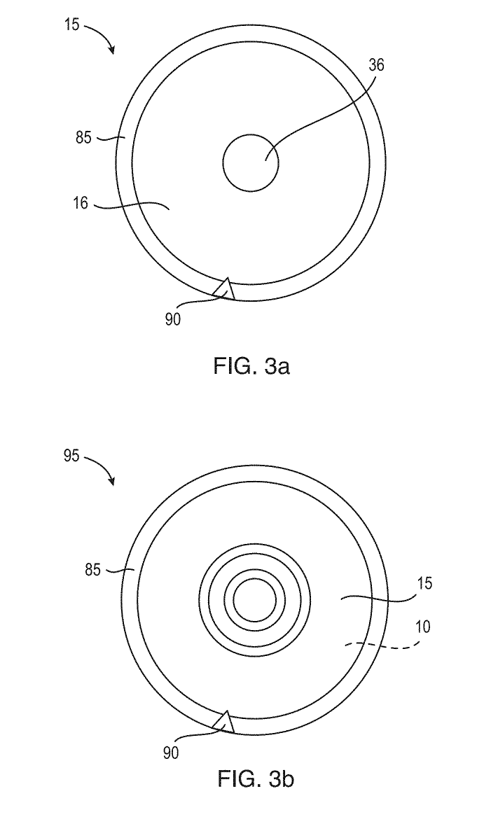

As set forth above, the disclosed system includes cover 15. The cover is sized and shaped to be secured over substrate assay region 45 to potentially lock down the position and pattern of analytes on substrate and to prevent contamination of the optical drive during testing. FIG. 3a illustrates one embodiment of cover 15 configured with body 16 of about the same size and shape as substrate 10. For example, the cover can be constructed to have about the same diameter and width as the substrate. As shown, the cover further includes center aperture 36 constructed with about the same size and shape of substrate center aperture 35. The cover is overlaid on substrate 10 as shown in FIG. 3b.

Cover 15 can be constructed from any material known or used in the art, including (but not limited to) polymeric material, such as polycarbonate, polyethylene, polyvinyl chloride (PVC), polystyrene, high impact polystyrene (HIPS), polypropylene, polyester, polyacryolonitrile (PAN), cellulose derivatives (such as ethylcellulose or cellulose acetate), methacrylate, polycarbonate, acrylic acid copolymer, acrylate, and combinations thereof. It should be appreciated that the materials used to construct cover 15 do not interfere with the signal return of the drive's laser and will likely be of similar material to that of the digital substrate.

The cover can be secured to the substrate using any method known or used in the art. For example, in some embodiments, peripheral outer ring 85 can be used. As shown in FIG. 3b, the outer ring is engaged around the outer circumference of both the substrate and the cover, thereby temporarily or permanently securing the cover to the substrate. In some embodiments, the outer ring can include a locking element (such as notch 90) that functions to secure the substrate and cover together during testing. In the example shown, the notch 90 is a small triangle shape that protrudes into the substrate circumference. The substrate itself has a complimentary empty triangle cut into the outer most edge that corresponds to the extruding notch.

The outer ring can be constructed from any desired material, such as one or more polymeric materials (e.g., nylon). The materials used to construct cover 15 do not interfere with the signal return of the DVD drive's laser.

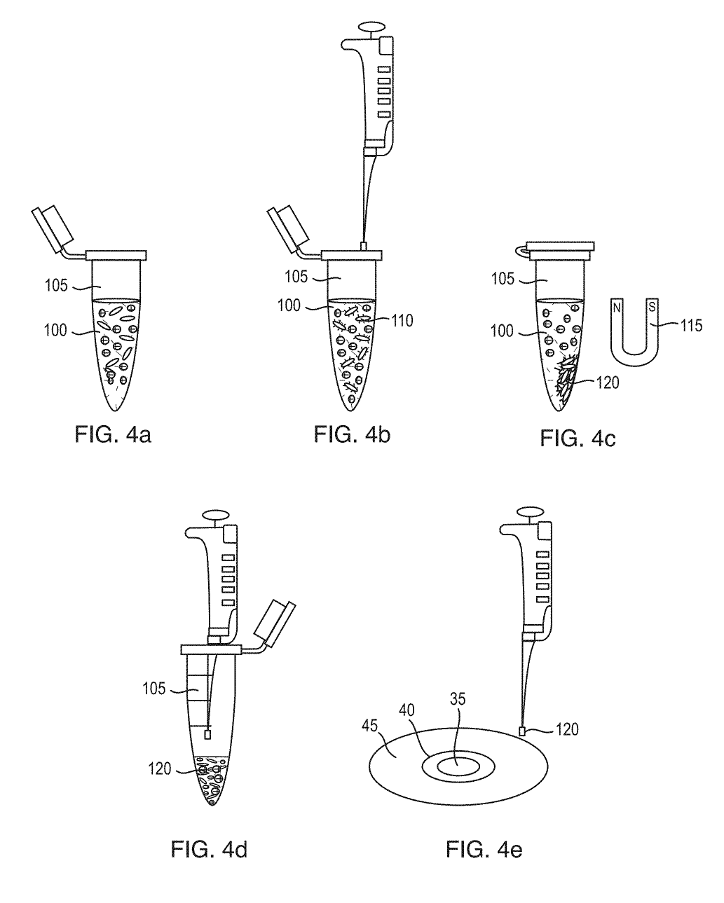

FIGS. 4a-4c illustrate an in vitro method of analyte preparation according to some embodiments of the presently disclosed subject matter. The term “analyte” as used herein refers to any chemical, biochemical, or biological entity that a user desires to detect in a sample. In some embodiments, the detected analyte can include one or more microbes, such as (but not limited to) bacteria, viruses, fungi, spores, and combinations thereof. As shown in FIG. 4a, a sample comprising analyte 100 is taken, for example, from a food, water or bodily fluid source suspected of being contaminated. In some embodiments, the sample is a liquid sample or is suspended in a liquid (such as water). Advantageously, only a small volume of sample is needed for the disclosed assay (e.g., 1 mL or less). The sample is deposited into collection tube 105 using standard techniques, such as pipetting.

In FIG. 4b, magnetic beads 110 coupled with a capture probe are added to the collection tube and are allowed to intermix with collected analyte sample 100. The term “capture probe” refers to a binding moiety with affinity for a target analyte. Suitable capture probes can include (but are not limited to) antibodies, antigens, polypeptides, enzymes, nucleic acids, aptamers, and combinations thereof. For example, antibodies to one or more analytes of interest can be immobilized on magnetic beads 110 using any known method. For example, one common practice is the use of Streptavidin coated nanobeads to bind with biotinylated antibodies or aptamers. The beads with the attached one or more capture probe interact and bind with the target analytes. In some embodiments, the magnetic beads can be blocked with bovine serum albumin (BSA) or other suitable blocking agents to prevent or reduce nonspecific binding.

Magnetic beads 110 can include any functionalized magnetic beads known or used in the art. For example, the beads can include ferromagnetic beads, paramagnetic beads, or combinations thereof. Ferromagnetic beads have the ability to attract magnets and can include beads constructed from iron, nickel, cobalt, tin, steel, and other alloys. Paramagnetic beads do not possess intrinsic magnetic activity when not exposed to a magnetic field, yet will become magnetized when exposed to a strong magnetic field. Suitable paramagnetic beads can include borosilicate glass nanobeads, dextran-coated nanobeads, polystyrene-magnetite and the like.

FIG. 4c illustrates magnetic separation of the beads with attached analyte from the suspension fluid using magnet 115. As shown, the magnetic beads are attracted to magnet 115 and thus are isolated in one portion of the collection tube. The supernatant comprising unbound analyte is removed using standard techniques, leaving the magnetic bead-bound analyte material 120 within collection tube 105. The bead-bound analyte material is re-suspended, removed from the collection tube, and deposited on assay region 45 of the substrate for testing, as shown in FIGS. 4d and 4e. It should be appreciated that more than one analyte can be isolated at a time using the detected method (e.g., each type of magnetic bead includes a unique capture probe to immobile a desired analyte).

Advantageously, a desired analyte is separated from the sample using a single collection tube 105. The method therefore avoids the need for a high-throughput device to perform assays and is beneficial where high volumes of samples exist and are of complex, heterogeneous solutions.

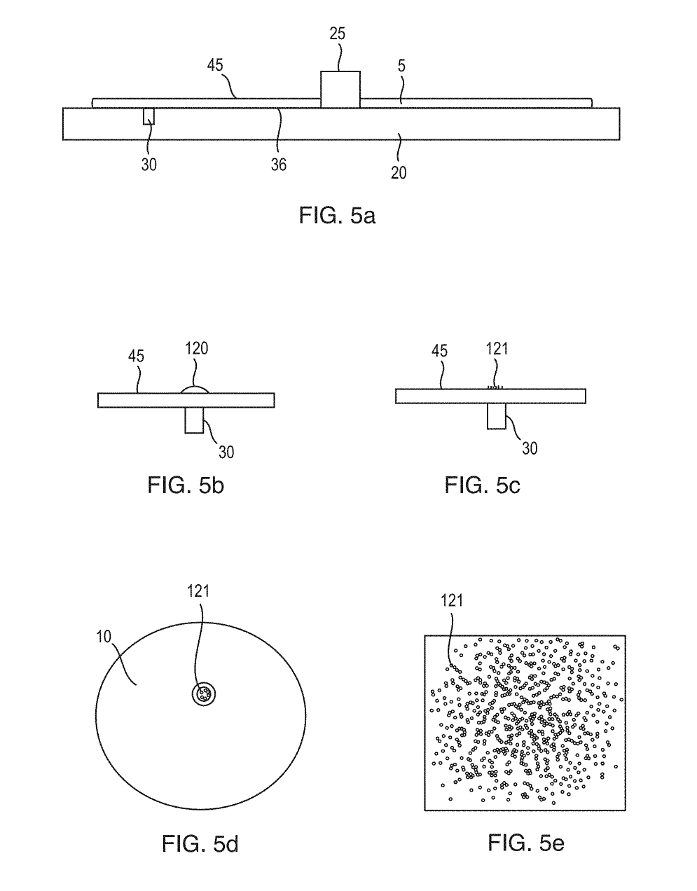

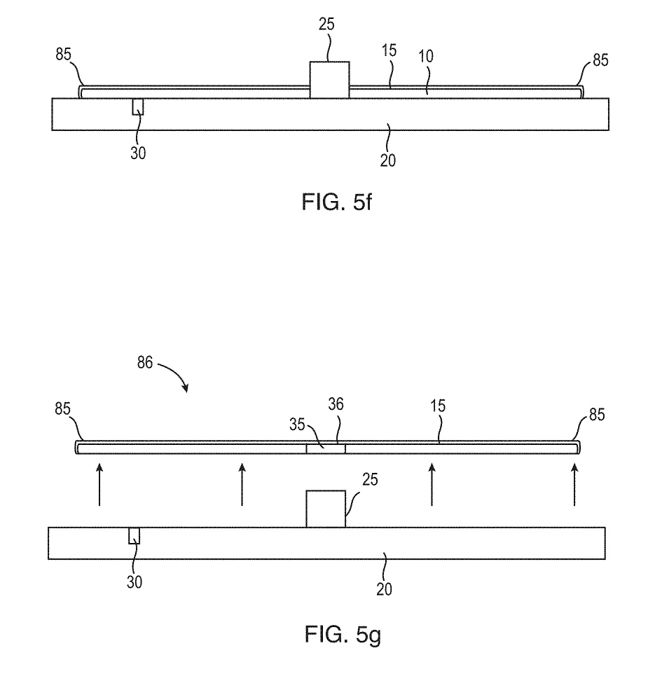

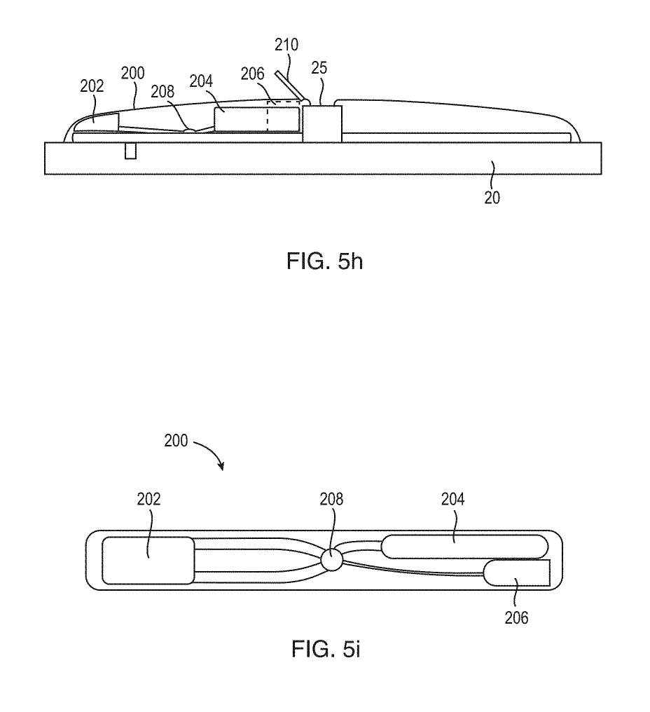

FIG. 5a illustrates one embodiment of substrate 10 positioned on base 20 with the assay region (e.g., data side) facing up, such that center aperture 35 fits over hub 25. As shown, base magnet 30 is positioned directly adjacent to one area of bottom face 36 of the substrate. In some embodiments, base magnet 30 can be an electric or fixed magnet. Electric magnets are those with magnetic fields due to electric current (e.g., the magnetic field is destroyed when electricity is stopped). Anything above 3.5 micro Tesla (mT) is sufficient, but the stronger the magnet pull, the faster the patterning occurs and the stronger the hold on the analytes during extraction of the fluid. One example of a suitable magnet is Neodymium at about 100 mT. In some embodiments, the base magnet can be adjustable to allow the user to select a desired magnetic strength.

Re-suspended magnetic bead analyte 120 is taken from the reacted sample and is deposited onto the surface of the digital substrate's data side (e.g., within assay region 45), as illustrated in FIG. 5b. Positioned directly below the application site is base magnet 30 that is shaped and oriented to attract the magnetic beads of the bound analytes. In this way, the analyte is attached to the assay region of the digital substrate. Any resuspension fluid is then removed, leaving condensed/patterned magnetic bead analyte(s) 121 deposited over the magnetic site, as shown in FIG. 5c. The beads are therefore attracted to the magnet, immobilizing the bound analyte in a specific area of the assay region of the substrate. Because the substrate is constructed from materials that are highly hydrophobic in nature (e.g., polycarbonate), the substrate rejects most types of fluids used for re-suspension of the analytes. The suspension fluid is therefore easily removed, such as by blotting, the use of compressed air, and/or simply agitating the substrate and shedding the resuspension fluid. In this way, the analytes are consolidated on the digital substrate, creating an assay assembly for an optical drive. Advantageously, the analytes within the fluid are culled and aggregated according to the shape and force of the magnetic field applied from below the digital substrate.

FIG. 5d illustrates a sample of analyte-bound magnetic beads 121 applied to substrate 10 having a high pull magnet 30 focused within the area of the sample. While still in solution, the magnetic beads separate and orient themselves into a spot (circled) corresponding to the diameter of the application site due to attraction to magnet 30. FIG. 5e illustrates the magnetic beads of FIG. 5d magnified to show the grouping.

FIG. 5f illustrates cover 15 positioned over substrate 10 to form an encapsulated assay assembly, with analyte-bound magnetic beads 121 located between the substrate and the cover. Outer ring 85 is secured about the circumference of the substrate and cover assembly, creating a sealed unit. Magnet 30 is then powered off to reduce the risk of sample shift. Substrate-cover assembly 86 is then removed from the base as a sealed and secure unit as indicated by the arrows of FIG. 5g. The assembly is inverted (e.g., the opposite orientation of that shown in FIG. 5g) and placed into a drive (e.g., a DVD drive) to run the assay. Inversely, the method described here could also function with the substrates reversed—the sample applied and concentrated on the cover and then the digital substrate is placed against the sample exposed cover.

Optionally, the system can include a macro or micro-fluidic cartridge clipped over the digital assay disk to collect, react, and deposit microbial samples. For example, as shown in FIG. 5h, cartridge 200 can include collection chamber 202 for housing the sample to be tested. The cartridge further can include chamber 204 for housing wash solution and sample reaction chamber 206. Chambers 202, 204, and 206 can all be routed to assay location 208 through the use of standard tubing and the like. In some embodiments, one or more chamber can be accessed by the user via lid 210 (e.g., a flip-top lid).

The presence of immobilized particles (microbes 80) on the substrate surface scatters light and prevents the optical drive from reading the data at that location within a particular assay region. As a result, “data read errors” are produced in proportion to the quantity of microbes present. The errors are counted and constitute the signal of the disclosed detection method. For example, data read errors can be detected using a Lite-On® DVD drive connected to a standard PC running the K-probe optical disc testing tool (K-probe V. 2.5.2) via the RSPC (Reed Solomon Product Code) currently used in DVD error correction.

Isolated analytes having the quality of magnetic attraction can be readily positioned and patterned on the substrate. Patterning in particular is essential for the success of low level sensitivity on the digital substrate. This owes to the necessity of positioning analytes in sectors of data over the data assay substrate that will result in RSPC, PI (Parity Inner), and PO (Parity Outer) or other recordable errors and corrections distinguishable from normal, more random, low level information. Bit errors will be corrected by the PI layer first if possible (when this happens, it is referred to as a Parity Inner Error (PIE)). If the number of bit errors becomes too large for the PI correction to handle, it results in a PI failure (PIF). The errors are then corrected in the PO layer. With the use of magnetically attracted analytes, target microbes can be immobilized on the surface (e.g., polycarbonate surface) of a digital substrate using a sufficient pull-strength magnet positioned on the opposite side of the disk. Because the substrate is highly hydrophobic in nature, the resuspension fluids used in the sample preparation methods are easily removed, leaving on the analytes on the substrate for detection after enclosing the system with the polycarbonate assay disk.

The visual graphing of the Parity Inner index (PI) (e.g., from K-probe tool) is useful in determining a prime area of the media upon which to deposit and pattern microbial samples. Data sector settings are useful for narrowing down the collection of data blocks to small data tracks 60 as opposed to a scan of an entire disk, thereby reducing scan time and increasing method sensitivity. The data from each scan can be manually or digitally saved and imported to a spreadsheet or database for further analysis. In some embodiments, the error counts per data track can be contrasted for a determination of change in the number of PI errors that (when increased) can be used to indicate positive detection and/or concentration.

Detection sensitivity can be maximized through the focused positioning of an analyte on the digital substrate at a location corresponding to predefined data sectors. Elution of the separated and concentrated samples is possible prior to analysis, the bound nanoparticles (e.g., paramagnetic nanobeads) can also be used to immobilize and pattern the target cells in precise geometries on substrate 10 for analysis. As a result, method detection levels can be improved and background noise can be reduced. Magnetic sorting and separation can be carried out at high throughput using a wide range of biological samples with minimal power requirements, without damaging the sorted entities and with all reagents condensed into a portable unit for better transportability and storage. The disclosed biosensor is beneficial for complex matrices, such as contaminated food and water samples, etc.

Advantageously, isolated analytes bound to paramagnetic nanobeads can be more readily positioned and patterned on the digital assay disk through the use of precisely oriented magnetic fields. The patterning enables the positioning of analytes over known data sectors on the substrate (e.g., DVD) surface, resulting in RSPC error corrections that are more readily distinguishable from random, low-level background data errors (e.g. improved detection sensitivity). Effectively, techniques common in microbial detection (such as agglutination) can be reproduced via paramagnetic nanobeads and powerful rare earth magnets to position microbes to enable their rapid detection. For example, Staphylococcus species, measuring <1 μm, can be positioned for detection on substrate 10. The scale of the unbound nanobeads is significantly smaller than the digital background structures. As a result, false positives are minimized.

Fundamentally, testing is performed using a simple two-step immuno-magnetic separation and optical drive detection approach. Thus, the separation process is effectively carried out in one step, followed immediately by detection, making the sample application process easier, faster, and less expensive than current detection methods.

Sample separation from a magnetic or paramagnetic bead analyte complex can yield high concentrations of isolated target analytes via magnetic separation. The method avoids the need for a high throughput device to perform assays and allows for all reagent to be condensed into a portable unit for better transportability and storage. The technique is beneficial where high volumes of samples exist and are of complex, heterogeneous solutions. Additionally, in instances of low sample volume, the separation of the analyte from the supernatant of the processed mixture can be accomplished directly on the detection substrate as opposed to doing it separately in vitro. This removes one step, make the sample application process easier and faster.

The disclosed biological detection system and method is advantageously based on familiar technology. Sample preparation is simplified and accessible, even to non-professional users.

Further, the disclosed system is cost-effective compared to prior art bio-detection systems, and is fast (requiring less than 1 hour to get a definitive result). For microbial pathogens, this detection method also eliminates the need for application through culturing, which is a common lab practice even today.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Testing of Paramagnetic Microspheres

Testing the immobilization and optical detection methods applied 2.3 μm and 0.7 μm paramagnetic microbeads (Bangs Laboratories) as surrogates for bacterial cells bound to paramagnetic nanobeads. Concentration, immobilization, and optical detection of paramagnetic microbeads were tested to validate the proposed detection method using optical drive technology. Microbeads were serially diluted in deionized water until the method's lower limit of detection (LLOD) was reached (defined as the concentration at which the signal-to-noise ratio falls below 1.8). Using 2.3 μm microbeads, this method produced a LLOD of 50 microspheres/5 mL initial sample. Testing of 0.7 μm microbeads at concentrations of 1000/5 mL was performed.

The steps used in the preparation are those described above and shown in FIGS. 4a-4e. Specifically, the sample is collected, magnetic-bead antibodies are added, the beads are separated using a magnet, and the analytes are re-suspended and applied to an assay disk. The assay is then performed and reported.

The re-suspended microbeads were collected from the separation stage (as described above) and were pipetted directly onto the surface of the data side of the digital substrate. This material had a robust polycarbonate surface that is highly hydrophobic in nature and naturally rejects most types of fluids used for re-suspension of analytes. A series of Neodymium magnets with additional structures were positioned directly below the application site. These structures shaped and oriented the magnetic analytes and held them to the digital substrate in a manner that yielded the highest detection levels in known data sectors of the DVD. FIGS. 2 and 3 illustrate the manner in which the microbeads collect and shape while within the magnetic field. The fluid used for re-suspension and application to the disk is then removed, either by blotting or simply by agitating the disk and shedding the excess material from the disk. The prepared assay/DVD is then placed into the optical disk drive to perform the assay.

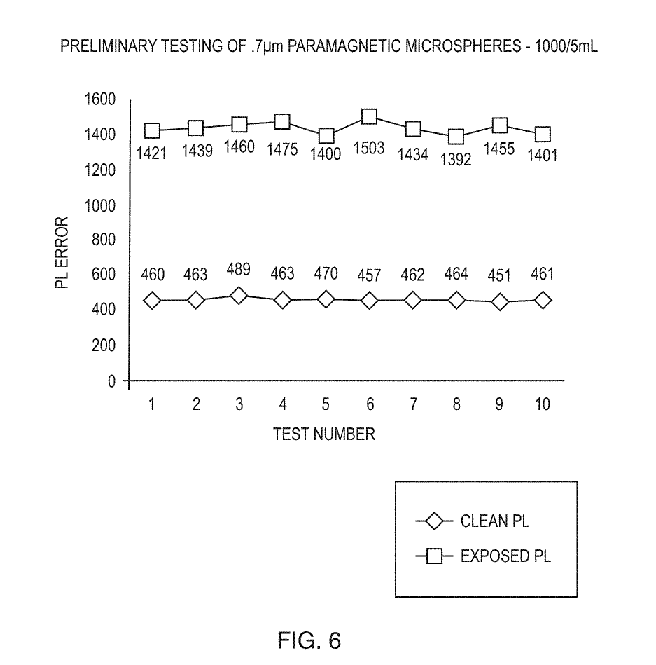

FIG. 6 illustrates the results of preliminary testing of 0.7 μm paramagnetic microbeads on the digital assay at 1000 beads per 5 mL initial sample size. As shown, consistent positive reads were obtained within about 2 minutes. The initial sample was concentrated to 2 μL for application using the disclosed magnetic separation and concentration method.

A clean PI is the number of Inner Parity corrections (to errors) in a given data sector of the digital medium. The exposed PI is the number taken from the same data sector after application of the sample. If there is no captured sample, the PI for clean versus exposed will remain about the same. In comparison, when there is a captured sample, the exposed PI number will increase in accordance with the number of captured analytes. FIG. 6 illustrates the difference between the lower (clean) PI values compared with the upper (exposed PI) values. In that study, the sample volume and the beat count were specifically made the same for each pass to examine for consistency (i.e., the same number of beads create the same level of increase in PI correction).

Further, testing has to date, tested over 20 separate methods in applying magnetism to the patterning substrate, ranging from multiple earth magnets to shaped electromagnets. FIGS. 5d-5e illustrate one example of this testing where a pointed earth magnet was positioned directly below a data sector the digital medium. For each of the magnetic patterning methods tested, sample sizes of ˜200 paramagnetic microbeads (2.3 μm diameter) per 5 mL were used. The initial sample was then reduced using the in vitro magnetic separation method. A collected sample of 2 μL was applied to the detection substrate (DVD) over the magnetic field of the magnets. For each assay method 10 rounds per concentration were collected until positive detection fell below 80%. The results indicate a 90% positive detection at a concentration of 50 paramagnetic microbeads (2.3 μm diameter) per 5 mL initial sample size.

Error correction on the DVD was performed by the RSPC, which treats data as arrays. It is reported that CIRC (from CDs) can correct about 500 bits of successive errors, whereas RSPC can correct about 2800 bits. The data on DVD discs was organized into sectors of 2048 bytes plus 12 bytes of header data. Blocks of 16 sectors were error-protected using RSPC, which was block oriented and more suitable for re-writable discs (with packet writing). The PI and PO (Parity Inner/Parity Outer) data were parity bytes calculated horizontally and vertically over the data bytes. In current DVD systems, the decoder chip includes two frame buffer controllers that are a (182,172) row RS decoder and a (208,192) column RS decoder. The RS decoder determines error locations and error values, and this information is sent to the frame buffer controllers to update the frame buffer content.

Fundamentally, the decoder architecture of the DVD optical drive performs by: calculating the syndromes from the received codeword; computing the error locator polynomial and the error evaluator polynomial; finding the error locations; and computing error values.

### Example 2

### DVD Error Rate Versus Log E. coli Concentration

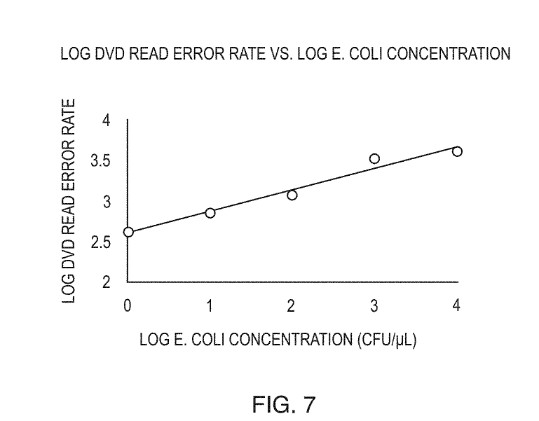

Additionally, individual tests were performed using a live, non-pathogenic, species of E. coli. These tests were performed using concentrated cells in an aqueous mixture to examine the functional effect cells have in the optical disk drive. Recent work has shown that bacterial cells can be detected on the surface of a modified DVD in less than 5 minutes. Bacterial cells have a length-scale on the same order of magnitude as that of the data pits read by an optical DVD drive, and scatter the laser light used to read the data pits, producing “read errors” corresponding to the location and number of the cells (FIG. 7). Note: log read error rate for distilled water blank was 1.9. Each sample read was completed in approximately 5 min. Again, 2 μL samples were applied directly to the digital substrate, enclosed, and the assay was run in a similar fashion to the method applying magnetic beads, described above.

E. coli cells were allowed to grow in medium of BSA and PBST in a 5 ml sample plate over a 5-hour period. Initial concentration of this culture was measured at 5000 cells per mL, or 5 cells per 1 μL. A single 2 μL application was pipetted onto the digital substrate 4e in a known data sector location of the digital medium 45 at zero hour. The disk was then enclosed using the cover 15 and inserted into the optical drive. The PI value for the data sector of interest was recorded and the assembly was removed and washed thoroughly. This test was repeated each hour using the cultured E. coli cells from the same sample. As can be seen from FIG. 7, it was found that the rate of increase in PI values for each sequential test coincides with the logarithmic growth rate of the E. coli. cells.

### CONCLUSION

10 separate methods of applying magnetism to the detection substrate were tested, ranging from multiple earth magnets to shaped electromagnets. For each of the magnetic patterning methods tested a sample size of ˜200 paramagnetic microbeads (2.3 μm diameter) per 5 mL were used. The initial sample was then reduced using the in vitro magnetic separation method as described. A collected sample of 2 μL was applied to the detection substrate (DVD) over the magnetic field of the magnets below. For each assay method 10 rounds per concentration were tested until positive detection fell below 80%.

Additionally, a rapid test was performed using a live, non-pathogenic, species of E. coli. The test was performed solely using concentrated cells in an aqueous mixture and was designed to examine the functional effect cells had alone in the optical disk drive. 2 μL samples were applied directly to the digital substrate, enclosed, and the assay was run in a similar fashion to the method applying magnetic beads, described above.

The patterning method described above yielded a >90% positive detection at the concentration of 50 paramagnetic microbeads (2.3 μm diameter) per 5 mL initial sample size.

What is claimed is:

1. A digital biosensor for detecting the presence or amount of one or more analytes in a sample, the digital biosensor comprising:
an optically read digital substrate that includes a top face with a layer comprising a data path capable of being read by an optical drive incident upon the layer, wherein the data path is encoded with a baseline data that is static, and wherein the top face is defined by an assay surface used for sample deposition;
a cover overlaid on the top face of the digital substrate, wherein the cover is attached to the top face about the perimeter of the digital substrate;
wherein the digital biosensor is configured such that an amount of the one or more analytes can be positioned between the top face of the digital substrate and the cover; and
wherein the presence, amount, or both of the one or more analytes can be detected by an interruption or change to the baseline data being read from the digital substrate by a digital optical device.

2. The digital biosensor of claim 1, wherein the digital substrate is a digital optical disk.

3. The digital biosensor of claim 1, wherein the analyte is selected from the bacteria, viruses, fungi, spores, or combinations thereof.

4. The digital biosensor of claim 1, wherein the digital substrate is constructed from one or more hydrophobic polymers selected from polyethylene, polyvinyl chloride (PVC), polystyrene, high impact polystyrene (HIPS), polypropylene, polyester, polyacryolonitrile (PAN), ethylcellulose, cellulose acetate, methacrylate, polycarbonate, acrylic acid copolymer, acrylate, or combinations thereof.

5. The digital biosensor of claim 1, wherein the cover is constructed from polycarbonate.

6. The digital biosensor of claim 1, wherein the cover is attached to the top face via an outer ring positioned about the circumference thereof.

7. The digital biosensor of claim 1, wherein the top face of the digital substrate comprises a plurality of uniformly distributed data pits and lands.

8. The digital biosensor of claim 7, wherein the pits have a width of about 0.3 μm separated by lands with a width of about 0.7 μm.

9. A system for preparing a digital biosensor, the system comprising:
a base with a top face defined by a hub extending upwards therefrom and a magnet positioned adjacent to the top face, wherein the hub is configured to maintain a digital biosensor on the base by cooperating with the central apertures of a cover and the digital substrate;
wherein the digital biosensor comprises:
an optically read digital substrate that includes a top face with a layer comprising a data path capable of being read by an optical drive incident upon the layer, wherein the data path is encoded with a baseline data that is static, and wherein the top face is defined by an assay surface used for sample deposition;
a cover overlaid on the top face of the digital substrate, wherein the cover is attached to the top face about a circumference of the digital substrate.

10. The system of claim 9, wherein the magnet is selected from an electromagnet or a fixed magnet.

11. A method of detecting the presence or amount of an analyte in a liquid sample, the method comprising:
depositing a volume of the sample within a container;
adding a volume of magnetic beads with an attached capture probe to the container, wherein the capture probe is selected to bind the analyte;
contacting the exterior of the container with a magnet to immobilize the analyte-bound magnetic beads in one area of the container;
removing the excess liquid from the container, leaving the analyte-bound magnetic beads within the container;
removing the magnet from contact with the exterior of the container;
resuspending the analyte-bound magnetic beads;
positioning a digital substrate on a base with a top face defined by a hub extending upwards therefrom and a magnet positioned adjacent to the top face, wherein the hub is configured to maintain the digital substrate on the base by cooperating with the central apertures of a cover and the digital substrate;
depositing the resuspended analyte-bound magnetic beads onto a top face of the digital substrate, such that the magnet of the base immobilizes the analyte-bound magnetic beads;
removing the suspension fluid from the sample;
overlaying a cover on the top face of the digital substrate, wherein the cover is attached to the top face about a circumference of the digital substrate to thereby create a digital biosensor with analyte-bound magnetic beads positioned between the top face of the digital substrate and the cover; wherein the top face of the digital substrate comprises a data path capable of being read by an optical drive incident upon the layer, wherein the data path is encoded with a baseline data that is static;
removing the digital biosensor from the system;
reading the digital biosensor on an optical drive, wherein the presence or amount of the analyte is detected by an interruption or change to the baseline data being read from the digital substrate by the optical drive.

12. The method of claim 11, wherein the top face of the base further comprises a hub extending upwards therefrom, wherein the hub is configured to maintain the digital biosensor on the base.

13. The method of claim 11, wherein the magnetic beads are selected from ferromagnetic beads, paramagnetic beads, or combinations thereof.

14. The method of claim 11, wherein the analyte is selected from the bacteria, viruses, fungi, spores, or combinations thereof.

15. The method of claim 11, wherein the digital substrate is constructed from one or more hydrophobic polymers selected from polyethylene, polyvinyl chloride (PVC), polystyrene, high impact polystyrene (HIPS), polypropylene, polyester, polyacryolonitrile (PAN), ethylcellulose, cellulose acetate, methacrylate, polycarbonate, acrylic acid copolymer, acrylate, or combinations thereof.

16. The method of claim 11, wherein the digital substrate is a digital optical disk.

17. The method of claim 11, wherein detecting the presence or amount of an analyte in a liquid sample is performed in 24 hours or less.

18. The method of claim 11, wherein the system magnet is an electromagnet or a fixed magnet.
